(19) United States
(12) Patent Application Publication
(10) Pub. No.: US 2002/0183248 A1
(43) Pub. Date: Dec. 5, 2002

(54) Method of using lectins for prevention and treatment of skin diseases and disorders

(76) Inventors: Michael J. Oldham, Oxnard, CA (US); Howard C. Krivan, Santa Barbara, CA (US); Richard C. Potter, Seeley Lake, MT (US)

Correspondence Address: Vorys, Sater, Seymour and Pease LLP, Suite 1111, 1828 L Street, NW, Washington, DC, 20036-5104, US

(21) Appl. No.: 10/154,156

(22) Filed: May 24, 2002

Related U.S. Application Data
(63) Continuation of application No. 08/453,281, filed on May 30, 1995, now abandoned.

(51) Int. Cl. A61K 38/16; A61K 7/04; A61K 7/06; A61K 7/11

(52) U.S. Cl. 514/8; 424/70.13; 424/70.14; 424/61

(57) ABSTRACT

Diseases and disorders of dermal tissue such as the skin, hair and nails are treated or prevented by administering one or more lectins, capable of binding to the surface of pathogenic microorganisms inhabiting the hair, skin, and nails, or of binding to the superficial tissues that comprise hair, skin, and nails. The lectins may be administered topically or subcutaneously to a patient infected with pathogenic microorganisms or in danger of being exposed to such pathogens. Lectins that stimulate cell mitosis may also be administered to accelerate wound healing and restore the appearance of age-wrinkled skin. Lectins that coagulate blood can be administered to assist in stopping bleeding from skin lesions. The lectins may be applied to the skin in a pharmceutically acceptable vehicle.

### BACKGROUND OF THE INVENTION

1. Field of the Invention

This invention relates generally to methods of prevention and treatment of skin diseases and disorders and, more particularly, to the use of topical administration of lectins for prevention and treatment of skin diseases and disorders.

2. Description of the Prior Art

Skin diseases and disorders, including diseases and disorders of the hair and nails, are commonly caused in man and other animals by a variety of bacteria, fungi, and viruses. Frequently, these diseases and disorders develop into chronic conditions which are only partially responsive to conventional therapies. These therapies are often uncomfortable for the patient, leading to poor patient compliance with the therapy and resultant exacerbation of the skin disease or disorder. For certain diseases and disorders, there are no therapies at all. As a result, there has been a longstanding need for an improved method for safe and effective treatment and prevention of skin diseases and disorders.

One of the most common bacterial-related skin diseases is acne vulgaris, or acne. Acne is common in pubescent boys and girls as a result of androgenic hormones acting upon susceptible hair follicles. The sebaceous gland associated with the follicle enlarges and ultimately the follicle opening is sealed off, leading to formation of a keratinaceous cyst. Certain anaerobes may be trapped in these cysts, notably Propionibacterium acnes, which can then metabolize sebum to produce irritating free fatty acids. These acids lead to the inflammation and abscesses associated with acne. Propionibacterium granulosum and Pseudomonas aeruginosa are also associated with acne.

The most common therapy for acne is topical benzoyl peroxide which, although it is an effective antimicrobial and keratolytic, is noteworthy for its harsh and severely drying effect upon skin. As a result, patient compliance is a serious problem with this drug, inasmuch as a lasting therapeutic response typically requires diligent administration over a period of six weeks or so. Other therapeutic agents for acne, such as retinoic acid and coal tar, can also have unwanted side-effects.

There are several staphylococcal diseases of the skin, including impetigo, ecthyma, folliculitis, furuncles, carbuncles, and staphylococcal scalded skin syndrome (SSSS). Impetigo is a childhood pyoderma of the face and extremities characterized by the formation of localized crusty regions. Staphylococcus aureus is the usual cause, although sometimes a Group A β-hemolytic streptococcus (GABHS), such as Streptococcus pyogenes, is implicated. Ecthyma is an ulcerative form of impetigo. S. aureus also causes folliculitis, which is a pyoderma of the hair follicles and apocrine areas; Pseudomonas aeruginosa has also been implicated. Furuncles (boils) and carbuncles (clusters of furuncles with subcutaneous spread of infection) are also caused by S. aureus. SSSS usually occurs in young children or immunosuppressed patients and is characterized by crusted lesions which lead to peeling of the epidermis in large sheets. Group II coagulase-positive staphylococci are the cause. Systemic penicillin or other antibiotics are generally prescribed for these diseases. However, this therapy can be problematical because of increasing bacterial resistance as well as patient intolerance to these compounds.

Many other bacteria can cause skin diseases. Erysipelas is caused by GABHS; erythrasma is caused by Corynebacterium minutissimum; and erysipeloid is caused by Erysipelothrix rhusiopathiae, a gram-positive bacillus. These diseases are also generally treated by prescribing antibiotics, just as the staphylococcus-caused diseases, and, hence, the same bacterial resistance and patient intolerance problems arise.

Paronychial (nail) infections are usually caused by micrococci, Pseudomonas, or Proteus. Once again, the recognized treatment is with antibiotics.

Common body odor is a disorder arising from bacterial and yeast-mediated breakdown of the concentrated fatty sweat secreted by apocrine sweat glands. The resultant unsaturated fatty acids have a characteristic pungent odor. The predominant microbe responsible for body odor is the anaerobe Propionibacterium avidum. Although this problem can be satisfactorily addressed by modern deodorant and antiperspirant formulations, many people are sensitive to the active ingredients, usually hydrated aluminum chloride salts.

Many skin disorders are the result of fungal infection which can generally be classified as either dermatophyte (ringworm) or yeast infections.

Dermatophytes are fungi that can invade the stratum corneum of the skin or other keratinized tissues derived from the epidermis, such as hair and nails. They may cause infection at most skin sites, although the feet, groin, scalp, and nails are most commonly affected. Three genera of pathogenic fungi that cause dermatophytosis in humans are: Trichophyton, Microsporum, and Epidermophyton. Tinea corporis (ringworm of the body), characterized by the annular lesions from which the disease takes it name, is usually caused by T. rubrum, M. canis, and/or T. verrucosum. In the case of tinea capitis (ringworm of the scalp), the lesions are caused by T. tonsurans, but are not annular. Tinea pedis (athlete's foot), manifested by itching and scaling, is usually caused by either T. rubrum or T. mentagrophytes. The scratch dermatitis and lichenification associated with tinea cruris (jock itch) are usually the result of infection by T. rubrum or E. floccosum, although certain yeasts can also be involved. Tinea unguium (ringworm of the nails), which can lead to destruction of the nails, is generally caused by a Trichophyton species. Griseofulvin is prescribed as a systemic therapy for all of these “ringworm” dermatophytoses. However, some patients are sensitive to griseofulvin, which is a penicillin derivative, and its use is contraindicated for all pregnant women. Oral and topical imidazoles are also prescribed. The oral use of ketoconazole is hampered by the possibility of severe, or even fatal, liver toxicity. Topical imidazoles can be irritating to the skin and can induce allergic reactions. Increasing dermatophyte resistance to both griseofulvin and imidazoles has further limited the usefulness of these drugs.

The most important yeast infections are candidiasis, pityriasis (tinea versicolor), and seborrheic dermatitis.

Infections from Candida albicans are expressed as a variety of forms of candidiasis. The most common symptoms include well-demarcated erythematous patches which are pruritic and exudative. Small pustules rim the lesions and occur in the umbilicus, groin, gluteal folds (diaper rash), axillas, inframammary areas, nails (candidal paronychia), and between the toes and fingers. Vaginal candidiasis, which results in vaginal discharge and inflammation, is addressed in copending U.S. application Ser. No. 08/317,599, by some of the inventors of this application. Vaginal candidiasis can also lead to the infection of penile tissues, which is a skin condition treatable by this invention. Imidazoles, such as miconazole nitrate, are frequently prescribed for candidiasis, but such compounds can cause irritation, burning, maceration, and allergic contact dermatitis. Nystatin is also a preferred therapy. Although nystatin has no serious side effects, candidiasis frequently recurs subsequent to this or other therapies.

Pityriasis is common in young adults and is characterized by multiple scaly lesions on the chest, neck, and abdomen. The causative fungal organism is Malassezia furfur (Pityrosporum orbiculare). Pityriasis can also occur in the scalp and is caused by M. furfur and M. orbiculare. Selenium sulfide in shampoo form is the usual therapy. However, recurrence is almost universal.

Seborrheic dermatitis, which causes dandruff, is caused by several species of Malassezia. It is usually apparent as a pruritic dry or greasy scaling of the scalp. Selenium sulfide shampoo is recommended but, as with pityriasis, does not provide an effective cure.

Some other superficial fungal diseases include: tinea nigra, an infection of the palms and soles caused by Exophiala (Phaeoanellomyces) werneckii; white piedra, an infection of hair shafts caused by Trichosporon beigelii; and black piedra, an infection of hair shafts caused by Piedraia hortae. Conventional antifungals are prescribed for these conditions, with the same undesirable safety or efficacy consequences described previously.

Viral diseases of the skin include warts (verrucae) and various herpes infections.

The wart viruses (papovaviruses) are circular, double-stranded DNA having about 8000 base pairs. Warts are expressed in a variety of forms and locations on the body, including: plantar, palmar, mosaic, periungual, filiform, and flat. Removal is accomplished by means such as acid treatment, surgery, freezing, or cantharidin therapy. All of these treatments must be performed in a clinical setting and are frequently painful for the patient. Recurrence of warts occurs in about one-third of patients within a year of these treatments.

Genital wart infections are one of the most prevalent sexually transmitted diseases (STD). They are caused by human papilloma virus types 1, 2, 6, 11, 16, and 18. They may be removed by electrocauterization, freezing, or topical applications of acids, but no treatment is completely satisfactory.

Herpes simplex type 1 (HSV-1) is responsible for fever blisters, for which there is no quick, effective remedy. Herpes simplex type 2 (HSV-2) causes genital herpes, which is a highly infectious and widespread STD. Herpes zoster (shingles) is caused by the varicella-zoster virus. Oral acyclovir has been used with some success for herpes infections, but even early treatment does not resolve latent infections or prevent recurrences.

It is noteworthy that certain non-dermal diseases can be prevented by neutralizing the pathogenic vector while it remains on a dermal surface, prior to invading other bodily tissues. For example, syphilis, caused by the spirochete Treponema pallidum, is not usually classified as a skin disease, but the pathogen can be transmitted to superficial penile tissues as a result of intercourse. Currently, there are no adequate prophylactic compounds to deal with this circumstance. Similarly, measles (rubeola), caused by a paramyxovirus, and German measles (rubella), caused by an RNA virus, are not usually classified as skin diseases. However, these viruses are easily spread to other dermal areas on the body, whereupon new lesions will form, as a result of rubbing or scratching in an effort to relieve pruritis. There are no particular treatments for rubella or rubeola, other than symptomatic treatments, such as for pruritis. These treatments, such as calamine lotion, are usually of limited effect and-duration.

In addition to bacterial, fungal, and viral diseases, there are other traumas and maladies of the skin. For example, wrinkles arise from aging of the skin, particularly through photo-aging processes. Retinoic acid has been used with some success to deal with this difficult problem, but this therapy often leads to irritation and other undesirable side-effects. Surgery is commonly employed, but this is usually painful and carries uncertain results.

The skin is highly susceptible to lacerations, burns, and other wounds. The focus of most therapies for these traumas is to discourage infection by the use of antiseptic compounds while natural recovery takes place. However, the alternative strategy of accelerating natural healing has not been dealt with successfully. In the case of lacerations, including open, surgical incisions, there is a need for a safe, effective thrombogenic agent.

Thus, it is apparent that for many diseases and disorders of the skin, existing therapies are either ineffective or have undesirable side-effects and, for certain other conditions, there is no therapy available. Prophylactic methods are similarly deficient. Accordingly, there is a clear need for a new dermatological approach which will afford safe and effective treatments for both prophylaxis and therapy.

### SUMMARY OF THE INVENTION

This need for safer and more effective therapy and prophylaxis for diseases and disorders of the dermal tissues, i.e., skin, hair and nails, has now been alleviated by the method of this invention, according to which one or more lectins, capable of binding to the surface of pathogenic microorganisms inhabiting the hair, skin, and nails, or of binding to the superficial dermal tissues that comprise hair, skin, and nails, are administered topically or subcutaneously to a patient infected with such pathogens or in danger of being exposed to such pathogens.

The method of the invention also provides for the use of one or more lectins to stimulate cell mitosis and thereby promote dermal cellular growth to restore the smooth structure of wrinkled skin due to aging and to promote the healing of skin wounds.

The method of the invention also provides for the use of one or more lectins to agglutinate and thereby stanch the bleeding associated with skin lacerations and open, surgical incisions.

The lectins may be applied according to the method of the invention either neat or dispersed in a pharmaceutically acceptable vehicle.

Accordingly, it is an object of the invention to provide an improved method for preventing and/or treating dermatological infections caused by bacteria.

It is a further object to provide a method of prophylaxis and/or treatment for acne.

It is a further object to provide a method of prophylaxis and/or treatment for body odor.

It is a further object to provide a method for preventing and/or treating dermatological infections caused by species of Staphylococcus.

It is a further object to provide a method for preventing and/or treating dermatological infections caused by species of Streptococcus.

It is a further object to provide a method for preventing and/or treating dermatological infections caused by fungi.

It is a further object to provide a method for preventing and/or treating dermatological infections caused by dermatophytes.

It is a further object to provide a method for preventing and/or treating dermatological infections caused by species of Trichophyton, especially T. rubrum.

It is a further object to provide a method for preventing and/or treating dermatological infections caused by species of Microsporum.

It is a further object to provide a method for preventing and/or treating dermatological infections caused by species of Epidermophyton.

It is a further object to provide a method for preventing and/or treating dermatological infections caused by yeast.

It is a further object to provide a method for preventing and/or treating dermatological infections caused by Candida albicans.

It is a further object to provide a method for preventing and/or treating dermatological infections caused by species of Malassezia.

It is a further object to provide a method of prophylaxis and/or treatment for seborrheic dermatitis.

It is a further object to provide a method for preventing and/or treating dermatological infections caused by viruses.

It is a further object to provide a method for preventing and/or treating dermatological infections caused by papovaviruses.

It is a further object to provide a method for preventing and/or treating dermatological infections caused by human papilloma virus types 1, 2, 6, 11, 16, or 18.

It is a further object to provide a method for preventing and/or treating dermatological infections caused by herpes viruses.

It is a further object to provide a method for preventing and/or treating dermatological infections caused by HSV-1.

It is a further object to provide a method for preventing and/or treating dermatological infections caused by HSV-2.

It is a further object to provide a method for preventing and/or treating dermatological infections caused by varicella-zoster virus.

It is a further object to provide a method for preventing and/or treating rubeola.

It is a further object to provide a method for preventing and/or treating rubella.

It is a further object to provide a method of prophylaxis for human males against syphilis.

It is a further object to provide a method for binding, and thereby neutralizing, pathogenic microorganisms located on the skin, hair, or nails.

It is a further object to provide a method for binding target receptors on the skin, hair, or nails, thereby preventing invasion by pathogenic microorganisms.

It is a further object of the invention to provide an improved method for removing wrinkles which arise as a result of aging.

It is a further object of the invention to provide an improved method for promoting the healing of skin wounds, including burns.

It is a further object of the invention to provide an improved method for stopping the bleeding associated with skin lacerations and open, surgical incisions.

### DETAILED DESCRIPTION OF THE INVENTION AND PREFERRED EMBODIMENTS

Lectins are carbohydrate-binding proteins of non-immune origin that agglutinate cells or precipitate polysaccharides or glycoconjugates, i.e., proteins or lipids conjugated to oligo- or polysaccharides. They are widely distributed and have been isolated from both plant and animal sources. Their reactions with living cells are based on their ability to bind with antibody-like specificity to particular arrangements of the sugar residues that make up oligo- or polysaccharides.

The surfaces of eucaryotic cells contain numerous molecules of glycoproteins and glycolipids. Such glycoconjugates are found in the plasma membranes of cells of multicellular animals, including mammals and humans, as well as on the surfaces of single-celled eucaryotic organisms. Similarly, the cell walls and capsules of bacteria and the envelopes of viruses contain structural polysaccharides and/or glycoproteins. The carbohydrate moieties of these molecules which are displayed on the cell surfaces exhibit great variety in composition and structure that serves to distinguish the types of cells and to serve as a signal to other cells or materials which come into contact with the cell. For, example, variation in the carbohydrate moieties of glycoproteins and glycolipids in the plasma membrane of red blood cells serves as the basis for conventional blood typing. When lectins recognize and bind to certain carbohydrate moieties, they may serve to cross-link and agglutinate the cells bearing the binding groups, a property that earns for them the alternate name of agglutinins.

Furthermore, because the same sort of carbohydrate moieties often serve as attachment points for pathogens to bind to target cells and invade them, lectins may block infection of target cells by blocking the sites used by pathogens as recognition markers. The same type of specific binding occurs between sperm and egg in conception, and can be blocked by lectins. The binding ability of lectins may be very specific for certain mono- or oligosaccharides, allowing lectins to be used as a powerful tool for investigating the oligosaccharide epitopes on the surface of organisms or cells. Lectins can distinguish between blood cells of specific blood type, malignant from normal cells, and among species and genera of organisms. While glycoproteins, glycolipids, and bacterial cell walls and capsules are believed to be the main lectin-binding locations on the surfaces of cells, it is not excluded that carbohydrate moieties derived from other molecules or cellular structures may be displayed on the cell surface or that other lectin-binding structures may be targets for the lectins used in the method of this invention.

Current medical uses of lectins include distinguishing erythrocytes of different blood types (blood typing). More recently, lectins have been used ex vivo in depleting T-cells of patients undergoing bone marrow transplantation.

Among the microorganisms that are bound by certain lectins are infectious organisms such as bacteria, protozoa, fungi, and viruses. Lectins may be used to identify such microorganisms in vitro and are also capable of binding to them in vivo, thereby preventing them from infecting living cells. Human disease-causing organisms that can be bound by lectins include the organisms responsible for numerous sexually transmitted diseases (as described in copending U.S. application Ser. No. 08/317,599) and diseases of the oral and alimentary canal (as described in copending U.S. application Ser. No. 08/385,306), as well as Propionibacterium acnes, Propionibacterium granulosum, Pseudomonas aeruginosa, Staphylococcus aureus, Streptococcus pyogenes, Corynebacterium minutissimum, Erysipelothrix rhusiopathiae, various species of Proteus, Propionibacterium avidum, Trichophyton rubrum, Microsporum canis, Trichophyton verrucosum, Trichophyton tonsurans, Trichophyton mentagrophytes, Epidermophyton floccosum, Candida albicans, Malassezia furfur, Malassezia orbiculare, Exophiala (Phaeoanellomyces) werneckii, Trichosporon beigelii, Piedraia hortae, papovaviruses, human papilloma viruses, HSV-1, HSV-2, varicella-zoster virus, Treponema pallidum, the virus causing rubella, the virus causing rubeola, and others. Other infections and diseases in which the portal of entry or initial attachment is the skin, hair, or nails are also capable of being treated or prevented by administration of lectins according to this invention.

According to the invention, a dose of lectins effective for binding and agglutinating pathogenic microorganisms and/or blocking the recognition sites on target cells is administered to the skin, hair, or nails prophylactically or as therapy. Because of the specificity of lectins for certain microorganisms, a mixture of lectins can be chosen for their ability to bind or agglutinate specific pathogens.

Lectins also have mitogenic activity and can induce quiescent cells to grow and multiply. For example, lectins can stimulate mitosis in lymphocytes. It is suspected that most lectins of vegetable origin have this ability.

According to the invention, a dose of one or more lectins sufficient to induce cell mitosis in skin can be administered in areas of age-wrinkled skin so as to mitigate or eliminate the wrinkling. Alternatively, a dose of one or more lectins sufficient to induce cell mitosis in skin can be administered so as to promote wound healing.

Many lectins also have the ability to agglutinate (coagulate) blood because of their ability to bind to both erythrocytes and leukocytes. According to the invention, a dose of one or more lectins sufficient to coagulate blood can be administered to the area of a skin laceration or to open, surgical incisions in order to stanch bleeding.

A representative listing of lectins, the abbreviations by which they are referred to, and their sources are given in Table 1.

TABLE 1
Representative Lectins, Abbreviations & Sources

| | |
|---|---|
| AAP | Aaptos papillata (sponge) |
| AAnA | Anguilla anguilla (eel serum) |
| AAurA | Aleuria aurantia (orange peel fungus) |
| ABA | Agaricus bisporus (common mushroom) |
| ABrA | Amphicarpaea bracteata (hog-peanut) |
| AGT | Agardhiella tenera (red alga) |
| AL | Hippeastrum hybrid (amaryllis bulb) |
| APA | Abrus precatorius (Jequirity bean) |
| AS | Avena sativa (oat) |
| BDA | Bryonia dioica (white bryony) |
| BPA | Bauhinia purpurea alba (camel s foot tree) |
| CA | Colchicum autumnale (meadow saffron) |
| CAA | Caragana arborescens (Siberian pea tree) |
| CCA | Cancer antennarius (California or blue crab) |
| ConA | Canavalia ensiformis (jack bean) |
| CPA | Cicer arietinum (chickpea) |
| CSA | Cytisus scoparius (Scotch broom) |
| DBA | Dolichos biflorus (horse gram) |
| DSA | Datura stramonium (jimsonweed, thorn apple) |
| ECA | Erythrina cristagalli (coral tree) |
| ECorA | Erythrina corrallodendrin (coral tree) |
| EEA | Evonymus europaeus (spindle tree) |
| GNA | Galanthus nivalis (snowdrop bulb) |
| GSA-I/GSA-II | Griffonia simplicifolia (African legume) |
| HAA | Helix aspersa (garden snail) |
| HPA | Helix pomatia (Roman or edible snail) |
| JAC (Jacalin) | Atocarpus integrifolia (jackfruit) |
| LAA | Laburnum alpinum (golden chain) |
| LBA | Phaseolus lunatus (also limensis) (lima bean) |
| LCA (LcH) | Lens culinaris (lentil) |
| LEA | Lycopersicon esculentum (tomato) |
| LFA | Limax flavus (garden slug) |
| LIP (Limulin) | Limulus polyphemus (horseshoe crab) |
| LOA | Lathyrus odoratus (sweet pea) |
| LTA (LOTUS) | Lotus tetragonolobus (asparagus pea) |
| MAA | Maackla amurensis (maackla) |
| MIH | Mangifera indica (mango) |
| MPA | Maclura pomifera (Osage orange) |
| NPL (NPA) | Narcissus pseudonarcissus (daffodil) |
| PAA | Persea americana (avocado) |
| PHA (PHA-L) | Phaseolus vulgaris (red kidney bean) |
| PNA | Arachis hypogaea (peanut) |
| PSA | Pisum sativum (pea) |
| PTP | Ptilota plumosa (red alga) |
| PWA | Phytolacca americana (pokeweed) |
| PTAgalactose | Psophocarpus tetragonolobus (winged bean) |
| PTAgalNac | Psophocarpus tetragonolobus (winged bean) |
| RCA-I/RCA-II | Ricinus communis (castor bean) |
| RPA | Robinia pseudoacacia (black locust) |
| SBA | Glycine max (soybean) |
| SJA | Sophora japonica (Japanese pagoda tree) |
| SNA | Sambucus nigra (elderberry) |
| SOF | Soduim fragile (green alga) |
| STA | Solanum tuberosum (potato) |
| TKA | Trichosanthes kirilowii (China gourd) |
| TL | Tulipa gesneriana (tulip) |
| TMT | Tomentine (seaweed Codium tomentosum) |
| UEA-I/UEA-II | Ulex europaeus (gorse or furze seeds) |
| VAA | Viscum album (European mistletoe) |
| VFA | Vicia faba (fava bean) |
| VGA | Vicia graminea (herb) |
| VRA | Vigna radiata (mung bean) |
| VSA | Vicia sativa (vetch) |
| VVA | Vicia villosa (hairy vetch) |
| WFA | Wisteria floribunda (Japanese wisteria) |
| WGA | Triticum vulgaris (wheat germ) |
| suc-WGA | Succinylated WGA |

The choice of lectins for prophylaxis or treatment of a particular infection is determined, in part, by the lectin-binding properties of the pathogenic microorganism, which is a function of the composition of the particular oligosaccharide residues of the glycoproteins and glycolipids found on the external surface of the pathogen.

For example, Staphylococcus aureus can be bound by the lectins WGA (Davidson, S K et al, J Clin Microbiol 15: 547-53 (1982)), ConA (Reeder, N J et al, J Immunol 196: 334-40 (1971)), and LIP (Gilbride K J et al, Prog Clin Biol Res 29: 525-35 (1979)). WGA and ConA have a binding affinity for N-acetyl-D-glucosamine residues expressed on a surface (Doyle, R J, Lectin-Microorganism Interactions, Marcel-Dekker (New York), 43-55 (1994)), and strains of S. aureus are known to express such residues (Slifkin, M, Lectin-Microorganism Interactions, Marcel-Dekker (New York), 144-5 (1994)).

Candida albicans can be bound by the lectins ConA, LCA, and GSA-II (Dean, J W et al, J Biol Chem, 265: 12553-62 (1990). Each of these lectins has binding specificity for N-acetyl-D-glucosaminyl residues (Doyle, ibid.). These carbohydrate moieties have, in turn, been shown relevant for the binding of C. albicans (Ghannoum, M A et al, Candida albicans: cellular and molecular biology, Springer-Verlag (Heidelberg), 144-163 (1991)).

Herpes simplex viruses can be bound by the lectin HPA (Slifkin, M et al, J Clin Microbiol 27: 1036-39 (1989)). HPA can bind to residues of N-acetyl-D-galactosamine (Doyle, ibid.). N-acetyl-D-galactosamines represent a major class of oligosaccharide chains in viral envelope proteins.

Alternatively, a lectin can be selected for its ability to bind appropriately to a dermal tissue, thereby blocking the potential binding sites for pathogens; this technique has applicability for both prophylaxis and therapy.

The choice of lectins for stimulating mitosis for the purpose of mitigating age-induced wrinkles or promoting the healing of wounds, including burns, is determined by the tissue-binding properties of the lectin. Examples of mitogenic lectins include PHA (Nowell, P C, Cancer Res 20: 462-66 (1960)), SBA (Licastro, F et al, Lectins, Vol. III, Walter de Gruyter & Co. (Berlin), 293-302 (1983)), and TL (Kilpatrick, D C et al, Lectins-Biology, Biochemistry, Clinical Biochemistry, Vol. 7, Sigma Chemical Co. (St. Louis), 259-63 (1990)).

Many lectins are capable of agglutinating blood and are, therefore, useful for stopping the bleeding from superficial wounds and open surgical incisions by local, e.g., topical, administration to a bleeding lesion. ConA, WGA, and LCA are examples of lectins capable of agglutinating all types of human blood.

While the lectins discussed above and the organisms and conditions against which they are effective are representative of useful lectins according to the invention, it is to be understood that other lectins may be discovered which are also useful for these purposes.

The administration of lectins for these various dermal diseases and disorders will depend upon the particular condition and whether prophylaxis or therapy is required. In certain instances, a mixture of lectins is preferred. For example, a prophylactic product designed to protect against a variety of dermal diseases would contain a mixture of lectins selected for their ability to bind to certain dermal receptors and/or individual pathogens.

In some cases, a single lectin will suffice. As an example involving prophylaxis, when Treponema pallidum is transmitted to the superficial penile tissues as a result of intercourse, it can be neutralized by the prompt administration of a lectin, thereby preventing development of syphilis. The lectin SBA binds to Treponema pallidum (Fitzgerald, T J et al, Infect Immun 24: 261-68 (1979)) and is useful for this application. In this case, the lectin product is applied either immediately before or after intercourse. If, instead of binding directly to Treponema pallidum, the lectin is chosen so as to bind to the penile receptors sought by the pathogen, then the lectin is preferably administered prior to intercourse.

For acne and other conditions, a single lectin product (containing one or more lectins) will frequently be useful for both prophylaxis and therapy. In many cases, the course of administration will begin with a therapeutic dosage because the condition is already well-developed. Upon resolution of the condition, a maintenance dosage will be employed for prophylactic purposes. Sometimes, the therapeutic and prophylactic dosages will be equivalent.

Certain therapeutic regimens of the invention, in order to satisfactorily resolve a particular condition, will require the initial administration of one lectin product followed by another, different lectin product.

The lectins may be administered in a variety of forms for delivery to dermal surfaces, either topically or subcutaneously. Topical vehicles include creams, ointments, sprays, lotions, gels, solutions, foams, soap and non-soap bars, shampoos, rinses, and powders. Some of these forms may also be pre-impregnated into gauze or other sorptive coverings intended to be applied to the skin. Vehicles may be either aqueous or non-aqueous. Some vehicles may contain agents, e.g., natural or synthetic polymers, which form a dry, occlusive film when applied to the skin. Such polymers might include cellulose derivatives such as sodium carboxymethyl cellulose, methylcellulose, 2-hydroxyethyl cellulose; poly(vinylpyrrolidone); poly(acrylic acid) and salts thereof; and the like, as are known to those skilled in the art. Such films may have controlled delivery properties in order to provide a sustained delivery of lectin to the target organism or dermal receptor. Other vehicles, for either controlled or bolus delivery of lectins, will be apparent to one of ordinary skill in the art. The concentration or proportion of the lectin active ingredient in the dosage forms used in the method of the invention will vary widely depending on the particular application. It is even possible to use the lectins in neat form, i.e., as pure solids without admixture of any vehicle, e.g., as a dusting powder of finely divided lectins applied to the skin. When the lectins are applied in a vehicle or excipient, the concentration will be determined by the amount of lectin to be applied to the dermal tissues, among other factors. If a high concentration of lectins on the dermal tissues is required a dosage form such as a lotion, ointment, or the like having a high concentration of lectins, e.g., greater than 50% by weight may be used. If a lesser concentration of lectins on the dermal tissues is sufficient to achieve the therapeutic or prophylactic goal, a less concentrated formulation, e.g., less than 50% by weight can be used. It is also according to the invention to apply the lectins dispersed in a fugitive vehicle, e.g., a vehicle that is absorbed into the skin or a volatile vehicle such as water or a pharmaceutically acceptable volatile alcohol, which serves to disperse the lectins over the surface of the tissues to be treated and then evaporates or is absorbed by the skin to leave a coating of lectins on the surface of the tissues. Lectins dispersed in such a vehicle may be applied to the skin by manual distribution or by spraying and allowed to remain on the surface until the fugitive vehicle has disappeared leaving a deposit of lectins on the skin surface. Such vehicles may merely deposit the solid lectins on the skin surface or may also contain non-volatile ingredients that can serve to hold the lectins in place on the tissues after the fugitive vehicle has departed.

Duration and amount of dosage will be determined by the type and severity of condition, including the number of pathogens to be neutralized, and whether prophylaxis or therapy is intended. Dosage is also dependent upon the strength of binding between the lectin and the pathogen receptor or dermal receptor, on the binding constant for the interaction between the lectin and the receptors, and on the number of receptors that have to be saturated with lectin in order to produce an effective response. Dosage will also be affected by the bioavailability of the lectin to interact with the receptors. Accordingly, while the practitioner can gain some guidance as to an effective dose from the experimental determination of the binding effectiveness of a given lectin for a particular dermal condition, it must be expected that determination of an effective dose will involve some experimentation of the type that is entirely conventional in the development of pharmaceutical treatment for the skin diseases and disorders which are the subject of this disclosure.

### EXAMPLE

This example illustrates the binding of various lectins to Propionibacterium acnes, which is a principal organism involved in the development of lesions associated with acne vulgaris. P. acnes (ATCC 6919) was grown under anaerobic conditions at 37° C. for 3-4 days on blood agar plates containing 5% sheep blood. The bacteria were harvested with 0.01 M sodium phosphate buffer (pH 7.2) containing 0.15 M NaCl (PBS), washed twice with PBS, and suspended to a final optical density of 0.9 in sodium bicarbonate buffer, pH 9.5. Bacteria (100 mL) were added to flat-bottomed wells of polystyrene microtiter plates (Corning) and incubated at room temperature overnight. The coated plates were then washed three times with Hanks balanced salt solution supplemented with HEPES buffer containing 0.1% (v/v) Tween 20 (HBSST), pH 7.2, followed by the addition of 15 μg (150 μg/mL HBSST) of the appropriate biotinylated lectin. After two hours at ambient temperature, the wells were emptied and washed three times with HBSST. Bound biotinylated lectin was detected by the addition of 100 ng of streptavidin alkaline phosphatase (10 ng/μL), followed after one hour by washing the cells as above, followed by the addition of 100 μg of p-nitrophenol phosphate (1 mg/mL). Color production was quantified using a spectrophotometer at 405 nm.

Lectins were evaluated for their possible reactivity with immobilized P. acnes in vitro. The lectins LcH, STA, ConA, PSA, VFA, and MPA showed markedly strong binding to P. acnes, producing optical densities that were greater than 3.00. The lectins GNA, CPA, NPA, LEA, Jacalin, UEA, and BPA showed strong binding, while CAA, LAA, SBA, WFA, RPA, and LBA bound moderately with P. acnes in vitro. Other lectins reacted weakly with these bacteria. The experimental data are summarized in Table 2 below.

TABLE 2

| Lectin | Optical Density |
|---|---|
| LcH | >3.00 |
| STA | >3.00 |
| ConA | >3.00 |
| PSA | >3.00 |
| VFA | >3.00 |
| MPA | >3.00 |
| GNA | 2.16 |
| CPA | 1.65 |
| NPA | 1.42 |
| LEA | 1.32 |
| Jacalin | 1.20 |
| UEA | 1.15 |
| BPA | 1.12 |
| CAA | 0.91 |
| LAA | 0.87 |
| SBA | 0.76 |
| WFA | 0.70 |
| RPA | 0.63 |
| LBA | 0.54 |
| VVA | 0.45 |
| DSA | 0.43 |
| PHA | 0.43 |
| CSA | 0.42 |
| Lotus | 0.41 |
| ECA | 0.40 |
| HAA | 0.40 |
| PNA | 0.38 |
| ABA | 0.32 |
| MAA | 0.31 |
| WGA | 0.31 |
| SJA | 0.27 |
| suc-WGA | 0.26 |
| TKA | 0.26 |

The invention having now been described, it should be understood that it may be embodied in other specific forms or variations without departing from its spirit or essential characteristics. Accordingly, the embodiments described above are to be considered in all respects as illustrative and not restrictive, the scope of the invention being indicated by the appended claims rather than by the foregoing description, and all changes which come within the meaning and range of equivalency of the claims are intended to be embraced therein.

We claim:

1. A method of preventing and/or treating diseases and disorders of dermal tissue such as the skin, hair, or nails of humans and other animals caused by pathogenic microorganisms which comprises locally administering to a cutaneous site at least one lectin capable of interfering with the infective capability of a pathogenic microorganism toward dermal tissue, in an amount effective to diminish said infective capability of said microorganism.

2. The method of claim 1 wherein said lectin is administered prophylactically.

3. The method of claim 1 wherein said lectin is administered therapeutically.

4. The method of claim 1 wherein said lectin is administered topically.

5. The method of claim 1 wherein said lectin is administered subcutaneously.

6. The method of claim 1 wherein said lectin is administered neat.

7. The method of claim 1 wherein said lectin is dispersed in a pharmaceutically acceptable vehicle.

8. The method of claim 7 wherein said vehicle is a fugitive vehicle.

9. The method of claim 8 wherein said vehicle is a volatile vehicle.

10. The method of claim 7 wherein said vehicle is selected from the group consisting of creams, ointments, sprays, lotions, gels, solutions, foams, soap and non-soap bars, shampoos, rinses, and powders.

11. The method of claim 1 wherein said lectin is capable of binding to said microorganism.

12. The method of claim 1 wherein said lectin is capable of binding to said dermal tissue.

13. The method of claim 1 wherein a plurality of said lectins is administered.

14. The method of claim 1 wherein said microorganism is a bacterium.

15. The method of claim 14 wherein said bacterium is a species of Staphylococcus.

16. The method of claim 14 wherein said bacterium is a species of Streptococcus.

17. The method of claim 1 wherein said microorganism is a fungus.

18. The method of claim 17 wherein said fungus is a dermatophyte.

19. The method of claim 18 wherein said dermatophyte is a species of Trichophyton.

20. The method of claim 19 wherein said dermatophyte is Trichophyton rubrum.

21. The method of claim 18 wherein said dermatophyte is a species of Microsporum.

22. The method of claim 18 wherein said dermatophyte is a species of Epidermophyton.

23. The method of claim 17 wherein said fungus is a yeast.

24. The method of claim 23 wherein said yeast is a species of Malassezia.

25. The method of claim 23 wherein said yeast is Candida albicans.

26. The method of claim 1 wherein said microorganism is a virus.

27. The method of claim 26 wherein said virus is a papovavirus.

28. The method of claim 26 wherein said virus is human papilloma virus type 1, 2, 6, 11, 16, or 18.

29. The method of claim 26 wherein said virus is a herpes virus.

30. The method of claim 29 wherein said herpes virus is HSV-1.

31. The method of claim 29 wherein said herpes virus is HSV-2.

32. The method of claim 29 wherein said herpes virus is varicella-zoster.

33. The method of claim 1 wherein the disease is acne.

34. The method of claim 1 wherein the disorder is body odor.

35. The method of claim 1 wherein the disease is seborrheic dermatitis.

36. The method of claim 1 wherein the disease is rubella.

37. The method of claim 1 wherein the disease is rubeola.

38. The method of claim 1 wherein said lectin is selected from the group consisting of WGA, ConA, HPA, LIP, LCA, SBA, GSA-II, LcH, STA, PSA, VFA, MPA, GNA, CPA, NPA, LEA, Jacalin, UEA, BPA, CAA, LAA, WFA, RPA, and LBA.

39. The method of claim 38 wherein said lectin is selected from the group consisting of LcH, STA, ConA, PSA, VFA, MPA, GNA, CPA, NPA, LEA, Jacalin, UEA, BPA, CAA, LAA, WFA, RPA, LBA, WGA, and SBA.

40. The method of claim 38 wherein said lectin is selected from the group consisting of LcH, STA, ConA, PSA, VFA, MPA, GNA, CPA, NPA, LEA, Jacalin, UEA, BPA, WGA, and SBA.

41. The method of claim 38 wherein said lectin is selected from the group consisting of LcH, STA, ConA, PSA, VFA, MPA, WGA, and SBA.

42. A method of alleviating age-induced skin wrinkles in humans comprising locally administering to skin tissue afflicted with wrinkles at least one lectin capable of inducing cell mitosis in the tissues comprising the wrinkled area in an amount effective to decrease the prominence of said wrinkles.

43. The method of claim 42 wherein said lectin is administered neat.

44. The method of claim 42 wherein said lectin is dispersed in a pharmaceutically acceptable vehicle.

45. The method of claim 44 wherein said vehicle is a fugitive vehicle.

46. The method of claim 45 wherein said vehicle is a volatile vehicle.

47. The method of claim 44 wherein said vehicle is selected from the group consisting of creams, ointments, sprays, lotions, gels, solutions, foams, soap and non-soap bars, shampoos, rinses, and powders.

48. The method of claim 42 wherein said lectin is administered topically.

49. The method of claim 42 wherein said lectin is administered subcutaneously.

50. The method of claim 42 wherein a plurality of said lectins is administered.

51. The method of claim 42 wherein said lectin is selected from the group consisting of PHA, SBA, and TL.

52. A method of promoting the healing of dermal wounds and burns in humans and other animals comprising locally administering to injured skin tissue at least one lectin capable of inducing cell mitosis in said injured tissue in an amount effective to promote healing of said injured tissues.

53. The method of claim 52 wherein said lectin is administered neat.

54. The method of claim 52 wherein said lectin is dispersed in a pharmaceutically acceptable vehicle.

55. The method of claim 54 wherein said vehicle is a fugitive vehicle.

56. The method of claim 55 wherein said vehicle is a volatile vehicle.

57. The method of claim 54 wherein said vehicle is selected from the group consisting of creams, ointments, sprays, lotions, gels, solutions, foams, soap and non-soap bars, shampoos, rinses, and powders.

58. The method of claim 52 wherein said lectin is administered topically.

59. The method of claim 52 wherein said lectin is administered subcutaneously.

60. The method of claim 52 wherein a plurality of said lectins is administered.

61. The method of claim 52 wherein said lectin is selected from the group consisting of PHA, SBA, and TL.

62. A method of promoting the coagulation of blood from skin lacerations or open surgical incisions in humans and other animals comprising locally administering to a bleeding lesion at least one lectin capable of agglutinating blood or blood constituents in an amount effective to promote healing of said injured tissues

63. The method of claim 62 wherein said lectin is administered neat.

64. The method of claim 62 wherein said lectin is dispersed in a pharmaceutically acceptable vehicle.

65. The method of claim 64 wherein said vehicle is a fugitive vehicle.

66. The method of claim 65 wherein said vehicle is a volatile vehicle.

67. The method of claim 64 wherein said vehicle is selected from the group consisting of creams, ointments, sprays, lotions, gels, solutions, foams, soap and non-soap bars, shampoos, rinses, and powders.

68. The method of claim 62 wherein said lectin is administered topically.

69. The method of claim 62 wherein said lectin is administered subcutaneously.

70. The method of claim 62 wherein a plurality of said lectins is administered.

71. The method of claim 52 wherein said lectin is selected from the group consisting of ConA, WGA, and LCA.

72. A method of preventing syphilis in human males which comprises locally administering to the penis of a human male at least one lectin capable of binding to Treponema pallidum or to superficial receptor sites for Treponema pallidum on penile tissues, said lectin being effective for diminishing the infective capability of Treponema pallidum, and said lectin being administered in an amount effective to diminish said infective capability of T. pallidum.

73. The method of claim 72 wherein said lectin is administered neat.

74. The method of claim 72 wherein said lectin is dispersed in a pharmaceutically acceptable vehicle.

75. The method of claim 74 wherein said vehicle is a fugitive vehicle.

76. The method of claim 75 wherein said vehicle is a volatile vehicle.

77. The method of claim 74 wherein said vehicle is selected from the group consisting of creams, ointments, sprays, lotions, gels, solutions, foams, soap and non-soap bars, shampoos, rinses, and powders.

78. The method of claim 72 wherein said lectin is administered topically.

79. The method of claim 72 wherein said lectin is administered subcutaneously.

80. The method of claim 72 wherein a plurality of said lectins is administered.

81. The method of claim 72 wherein said lectin is SBA.

82. A pharmaceutical composition for administering lectins to the skin comprising at least one lectin dispersed in a pharmaceutically acceptable vehicle.

83. The composition of claim 82 wherein said composition comprises a plurality of lectins.

84. The composition of claim 82 wherein said vehicle is an aqueous vehicle.

85. The composition of claim 84 wherein said composition additionally comprises a film-forming polymer, whereby said polymer is deposited on said skin as a polymer film containing said lectin.

86. The composition of claim 85 wherein said polymer film is capable of sustained delivery of said lectins to the skin.

87. The composition of claim 82 wherein said vehicle is an emulsion comprising an organic phase, an aqueous phase containing at least one lectin, and an emulsifier.

88. The composition of claim 87 wherein said composition additionally comprises a film-forming polymer, whereby said polymer is deposited on said skin as a polymer film containing said lectin.

89. The composition of claim 88 wherein said polymer film is capable of sustained delivery of said lectins to the skin.
